(19) United States
(12) Patent Application Publication
(10) Pub. No.: US 2026/0062671 A1
(43) Pub. Date: Mar. 5, 2026

(54) METHOD AND DEVICE FOR TREATING AN ADIPOSE TISSUE

(71) Applicants: Maria Cristina COLLIVIGNARELLI, MORTARA (PV) (IT); Andrea POGGIO, Sesto San Giovanni (MI) (IT); Antonello TATEO, PAVIA (PV) (IT)

(72) Inventors: Maria Cristina COLLIVIGNARELLI, MORTARA (PV) (IT); Andrea POGGIO, Sesto San Giovanni (MI) (IT); Antonello TATEO, PAVIA (PV) (IT)

(21) Appl. No.: 19/106,940

(22) PCT Filed: Aug. 2, 2023
(86) PCT No.: PCT/IT2023/050189

§ 371 Date: Feb. 26, 2025

(30) Foreign Application Priority Data
Sep. 5, 2022 (IT) 102022000018105

(51) Int. Cl. C12N 5/0797; A61K 35/35; B01D 61/14; C12N 5/077

(52) U.S. Cl. CPC C12N 5/0623; A61K 35/35; B01D 61/1471; C12N 5/0653; B01D 2239/1216; C12N 2509/10

(57) ABSTRACT

The invention concerns a method, and a device that can implement this method, adapted to obtain adipose based compounds, of selectively variable particle sizes, distinguished by high levels of purity and by high concentrations of viable mesenchymal stem cells, which can be used in autologous transplantation procedures mainly for regenerative purposes, as well as for filling and/or volumizing purposes.

### TECHNICAL FIELD

The invention pertains to the field of methods and devices for the production of adipose based compounds destined for reimplantation in the same donor patients with a single-stage procedure, both with surgical and outpatient methods.

More in detail, the invention relates to a method and a device adapted to obtain adipose based compounds, of selectively variable particle sizes, distinguished by high levels of purity and by high concentrations of viable mesenchymal stem cells, which can be used in autologous transplantation procedures mainly for regenerative purposes, as well as for filling and/or volumizing purposes.

### BACKGROUND ART

Regenerative medicine defines an immense biomedical and clinical field, which comprises numerous medical and surgical specializations all having the purpose of treating degenerative diseases or aging phenomena, and of regenerating organs and tissue.

Recent development in research in this field derives from the therapeutic potential that can be foreseen in the use of numerous factors with biological activity and of cells with direct regenerative action, i.e., capable of differentiating in the receiving tissue/organ (paracrine), and capable of awakening the regenerative capacity of local cells collected from the same subject.

After experience gained on the placenta and bone marrow, to date, it has been seen how adipose tissue, commonly available and easily collectable, forms the greatest reserve of regenerative elements and of MSC (Mesenchymal Stem Cells). These are undifferentiated and multipotent cells that have not yet acquired a specific function and that maintain the capacity to direct their evolution. Thanks to their therapeutic potential, which includes inhibition of inflammation and of apoptosis, angiogenesis stimulation, recruitment and the above-mentioned capacity for differentiation, these cells form the basis of regenerative medicine.

The development of regenerative medicine is merged with the practice of lipofilling, i.e., a very common mini-invasive adipose fat grafting surgical procedure, having numerous applications in reconstructive and aesthetic plastic surgery. With such practice, adipose tissue has been used for a long time exclusively for volumizing purposes, although showing a high variability of engraftment and with unpredictable or, at times, no results. All this was before knowing about its regenerative biological potential.

To date, the lack of standardized and widely shared protocols, both with regard to collection and to processing and transplantation, is considered the greatest critical point of adipose grafting procedures. Recent discoveries in the field of tissue regeneration have given a considerable impetus to the research and development of new methods, not only with the intention of acquiring better knowledge of the composition of the adipose tissue collected, but also with the intention of optimizing both adipose engraftment and regenerative biological activity of the grafted product.

To date, high concentrations of stem cells can be obtained from a Stromal Vascular Fraction (SVF), through enzymatic digestion, or from adipose tissue, through mechanical processing of the fat contained therein.

The enzymatic digestion method has important negative aspects as it is only permitted by Regulatory Bodies (AIFA, EMA, FDA) for research purposes and, moreover, using a laboratory procedure to be carried out in the cleanroom, which determines delayed implantation on the patient after collection. Added to this is the further important negative aspect due to the fact that numerous recent studies have shown a decreased activity of the stem cells obtained with this method.

The mechanical extraction method is instead permitted by the Regulatory Bodies and, moreover, as it can be being performed at the time of collection in the operating theatre or in the outpatient's surgery, making it possible to implant the stem cells thus obtained immediately after collection of the original adipose tissue.

Currently, the main scientific literature is oriented toward the use of the whole of the adipose structure collected, according to the concept of Nanofat, in which fragmentation is below 600 microns. This determines the important limitation of excluding the use of products with higher fragmentations, such as Millifat and Microfat, except for greater volumes.

Excluding centrifugation solutions, which are being slowly abandoned, current mechanical extraction solutions have some important negative aspects:
they require manual compression forces for the passage and forced homogenization of the adipose tissue collected, with numerous passages through the syringe via different connectors, which gives rise to high cell stress and a reduction of the viability and of the survivability of the stem cells thus obtained, and the formation of new oily residues caused by the rupture of adipocytes;
they are dependent on the operator, who can apply different manual compression forces on the adipose tissue collected;
they require multiple passages, from syringe to syringe, of the adipose tissue collected, which results in an increase in the probability of contamination of the final product;
the liquid residues can only be removed from the final product through decantation;
fibrous and/or oily and/or haematic residues in the final product are not disposed of.

### Presentation of the Invention

The object of the invention is to propose a method, and a device that can implement this method, capable of overcoming the aforesaid limits and drawbacks, of the current methods for extracting stem cells from adipose tissue, thereby facilitating the attainment of adipose based compounds, of selectively variable particle size, marked by high levels of purity and by high concentrations of viable mesenchymal stem cells.

The object of the invention is also to propose a method that has the following characteristics:
maximum standardization of the fat processing procedure, in direct relation with the collection procedure;
maximum standardization and constant reproducibility of the result;
execution in a closed circuit or a circuit that guarantees a condition of maximum asepsis;
independence from the operator;
elimination of fibrous and/or oily and/or haematic residues from the stem cells obtained;
concentration of the stem cells obtained;
maximum reduction of cell stress to preserve the viability of the stem cells obtained;
selection of the particle size of the final product;
removal of excess liquids from the final product.

Among the aforesaid characteristics, elimination of residues and reduction of cell stress of the final product are fundamental, as impurities and cell stress greatly reduce the survival of Mesenchymal Stem Cells (MSC).

The object of the invention is achieved with a method for treating an adipose tissue according to the principal independent claim 1.

The invention also relates to a device for treating an adipose tissue according to the independent claim 6.

Further features of the method and of the device of the invention are described in the dependent claims.

The method and the device of the invention produce numerous and important advantages, as:
the sequence of treatments of the adipose tissue collected, providing a first physical-mechanical treatment step and a second first physical treatment step, allows complete objectivity and complete repeatability of the treatment implemented;
by means of a filtering column and a vacuum dehydration-filter apparatus associable therewith, both the treatments steps implemented can be executed very easily;
by means of said filtering column and said vacuum dehydration-filter apparatus, the plant layout configuration is a very simple, and does not require particularly specialized operators for its use;
by means of said filtering column and said vacuum dehydration-filter apparatus, the plant layout configuration is adaptable both for recovery of the Nanofat (which is also treated by the vacuum dehydration-filter), and of the Microfat and Millifat (which are treated only by the filtering column), so that the treatment implemented is highly applicable and resilient;
by means of a radial stirrer means of oscillating type associable with said filtering column, a vibrating sieving treatment is implemented, avoiding the transmission of sussultory movements, traction, compression, or centrifugation to the treated adipose tissue, so as to reduce its mechanical stress and consequently increase its viability and the survivability of the stem cells obtained therefrom.

Other detailed aspects that give further advantages to the invention are:
the total duration of the enhancement process of the adipose tissue is less than 5 minutes: thus limited time has the large advantage of reducing the stress to the adipose tissue treated is subjected to a minimum;
the protective synergic action of the hyaluronic acid: the great strength of the technology proposed is the minimization of any mechanical compression/expansion stress on the cell material during particle size selection, and the hyaluronic acid, given its important viscoelastic property, has the capacity to cooperate with the process forming a protective film that helps the cell material to pass through the metal mesh, greatly reducing stress;
the vacuum filtration process has the important aim of extracting excess water present in the Nanofat fraction: this treatment is useful and necessary because, as indicated in the international scientific literature, removal of the cell material of adipose tissue causes the release of an extracellular water content that in this step is free and therefore must be eliminated.

### BRIEF DESCRIPTION OF THE DRAWINGS

Further features and advantages of the invention will be more apparent from the more detailed description set forth below, with the aid of the drawings, which show preferred embodiments thereof, illustrated by way of non-limiting example, wherein:

FIG. 1 shows, in a schematic flow diagram, all of the steps of implementation of a method for treating an adipose tissue according to the invention;

FIG. 2 shows, in a schematic flow diagram, a possible variant of embodiment of the aforesaid method;

FIGS. 3 and 4 schematically show the plant layout of a device for implementation of the aforesaid method and of its possible variants of embodiment;

FIGS. 5, 6, and 7 show some graphs relating to experimental results obtained using the method and the device of the invention.

### DETAILED DESCRIPTION OF THE INVENTION

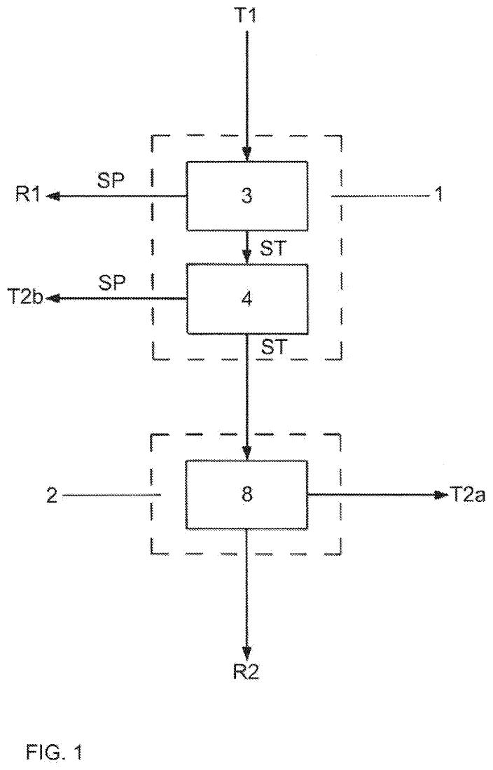

With reference to the details of FIG. 1, a method for treating an adipose tissue according to the invention, substantially comprises the steps of:
providing a plurality of sieves with progressively decreasing mesh sizes;
providing radial stirrer means of oscillating type;
providing vacuum dehydration-filter means;
collecting an adipose tissue T1 from a donor patient;
subjecting said adipose tissue T1 to a first physical-mechanical treatment step by means of said plurality of sieves with progressively decreasing mesh size and, by means of said radial stirrer means of oscillating type, separating at least an oversize fraction containing fibrous fractions of different particle sizes and a remaining wet and homogenized undersize fraction, of Nanofat type, rich in stem cells;
subjecting at least said remaining wet and homogenized undersize fraction to a second physical treatment step, by means of said vacuum dehydration-filter means, to eliminate the water, in order to concentrate the same fraction and obtain an adipose tissue T2a of Nanofat particle size, without fibrous and/or oleic and/or haematic residues R1 and aqueous residues R2, containing viable mesenchymal stem cells in a high concentration, where said adipose tissue T2a is adapted to be destined for reimplantation in the same donor patient.

In particular, the first physical-mechanical treatment step comprises the steps of:
providing a filtering column 1, provided with at least a first and a second sieve 3, 4 placed in sequence, with mesh of decreasing size according to the direction of the filtering flow;
operating said filtering column 1 by means of said radial stirrer means of oscillating type;
carrying out an impurity separation and homogenization treatment on the adipose tissue T1 by means of the first sieve 3, obtaining an oversize SP and an undersize ST;
disposing of the fibrous and/or oleic and/or haematic residues R1 contained in the oversize SP of the first sieve 3;
carrying out an impurity separation and homogenization treatment, by means of the second sieve 4, on the undersize ST of the sieve immediately prior to it to obtain an oversize fraction T2b and a wet undersize fraction ST comprising Nanofat adipose tissue T2a and aqueous residues R2.

In particular, the second physical treatment step in turn comprises the steps of:
providing a vacuum dehydration-filter apparatus 2, provided with a filter 8;
providing a suction pump 9 adapted to operate said vacuum dehydration-filter apparatus;
carrying out a separation treatment of aqueous residues R2 from said remaining wet and homogenized undersize fraction ST of the second sieve 4 by means of the filter 8 and the suction pump 9 of the vacuum dehydration-filter apparatus 2, so as to obtain an adipose tissue T2a of Nanofat particle size, also without aqueous residues R2.

With reference to the details of FIG. 1, a method for treating an adipose tissue, according to a first variant of the invention, comprises the further step of:
collecting the oversize SP of the second sieve 4, comprising an adipose tissue T2b of Microfat particle size, naturally with a low water content and without fibrous and/or oleic and/or haematic residues R1, containing viable mesenchymal stem cells in a high concentration, where said adipose tissue T2b of Microfat particle size is adapted to be destined for reimplantation in the same donor patient.

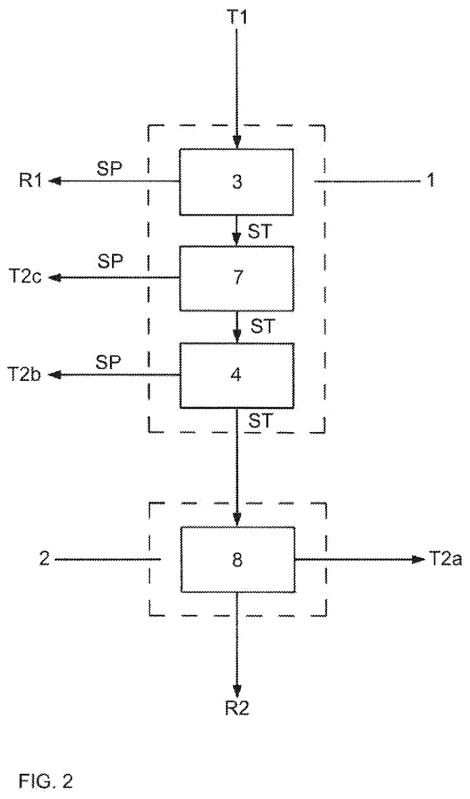

With reference to the details of FIG. 2, a method for treating an adipose tissue, according to a further variant of the invention, comprises the further steps of:
providing a third sieve 7, interposed between the first and the second sieve 3, 4 obtaining a further oversize and a further undersize;
by means of the third sieve 7, carrying out an impurity separation and a subsequent homogenization treatment on the undersize ST of the sieve immediately prior to it;
collecting the oversize SP of the third sieve 7, so as to obtain an adipose tissue T2c with Millifat particle size, naturally with a low water content and without fibrous and/or oleic and/or haematic residues R1, containing viable mesenchymal stem cells in a high concentration, destined for reimplantation in the same donor patient.

### Detailed Description of a Device for Implementation of the Method of the Invention

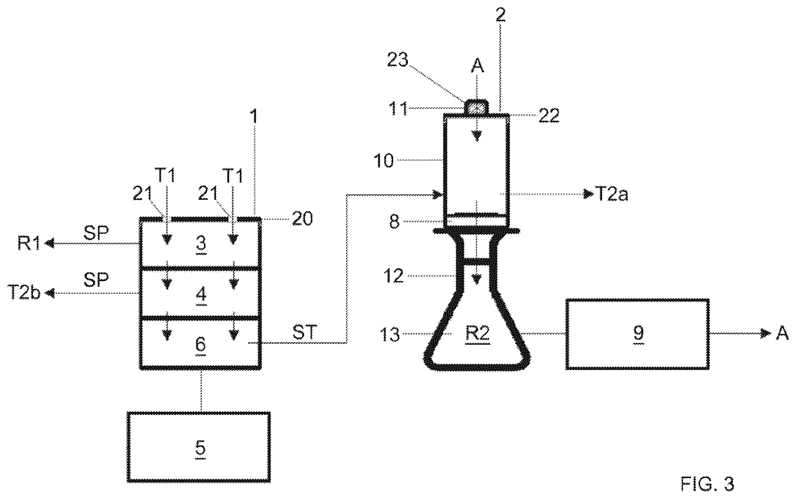

With reference to the details of FIG. 3, a device for treating an adipose tissue T1, collected from a donor patient, according to the invention, substantially comprises:
a filtering column 1, provided with at least a first and a second sieve 3, 4, with mesh of decreasing size according to the direction of the filtering flow, adapted to be operated by radial stirrer means 5 of oscillating type
a vacuum dehydration-filter apparatus 2, provided with a filter 8, adapted to be operated by a suction pump 9,
   where said filtering column 1, by means of said second sieve 4, allows an undersize comprising a wet adipose tissue of Nanofat particle size and an oversize comprising an adipose tissue T2b of Microfat particle size, naturally with a low water content, to be obtained and where said vacuum dehydration-filter apparatus 2 allows an adipose tissue T2a without aqueous residues R2 to be obtained, where each adipose tissue T2a, T2b is without fibrous and/or oleic and/or haematic residues R1, contains viable mesenchymal stem cells in a high concentration, and at least one of these is destined for reimplantation in the same donor patient.

The first and the second sieve 3, 4 respectively define the top and the base of the filtering column 1.

Preferably, said first and second sieve 3, 4 comprise filtering mesh of 2 mm and of 0.6 mm in size, respectively.

The filtering column 1 further comprises:
a reversible closing cover 20 of the first sieve 3, provided with sealed points 21 for insertion of the adipose tissue T1 optionally in combination and/or dosage with substances having therapeutic and synergic action, such as hyaluronic acid, etc.;
a collection chamber 6 of the undersize ST of the second sieve 4, equipped with a flat bottom or with a weak slope.

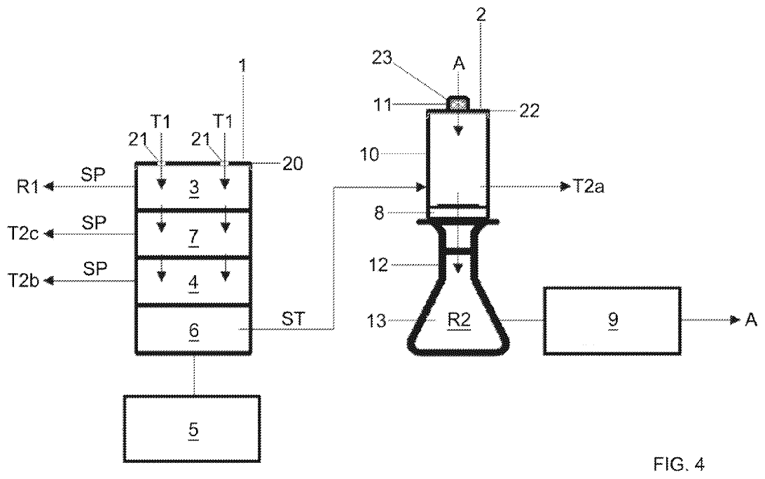

According to a more complex possible embodiment, shown in FIG. 4, the filtering column 1 can further comprise:
a third sieve 7, arranged in intermediate position between the first and the second sieve 3, 4,
   so as to allow a further oversize to be obtained comprising an adipose tissue T2c of Millifat particle size, naturally with a low water content and without fibrous and/or oleic and/or haematic residues R1, containing viable mesenchymal stem cells in a high concentration, destined for reimplantation in the same donor patient.

The third sieve 7 defines the central portion of the filtering column 1.

Preferably, said third sieve 7 has a mesh size of 1 mm.

The stirring means of the sieves 3, 4 and 7 of the filtering column 1 comprise a radial mixer 5, for example of Vortex Mixer type, adapted to create a movement that is oscillating and not sussultory, to obtain a sort of vibrating sieving.

The vacuum dehydration-filter apparatus 2 further comprises:
an upper section 10, provided with a reversible closing cover 22;
an atmospheric air A inlet 11, positioned on the closing cover;
one-way filtering valve means 23 associated with the inlet 11, adapted to filter atmospheric air A;
a lower section 12, provided with a tank 13 for collecting aqueous residues R2, separated from the undersize ST of the second sieve (4) of the filtering column 1.

Preferably, the filter 8 of the vacuum dehydration-filter apparatus 2 is made of paper or of steel, with particle size between 20 and 25 micron.

### Example 1

The treatment according to the invention was tested in the laboratory in order to verify enhancement of the useful and “valuable” fractions T2a, T2b, T2c (containing viable mesenchymal stem cells) of an adipose tissue T1, obtained by means of a physical-mechanical apparatus, consisting of a filtering column 1, having the aim of eliminating the fibrous and/o oleic and/o haematic part and of homogenizing the remaining part of said adipose tissue T1, and of a physical apparatus, consisting of a vacuum dehydration-filter 2, having the aim or eliminating the water present in said adipose tissue T1 so as to concentrate said valuable fraction T2a.

The filtering column 1 was configured with two sieves 3, 4 or with three sieves 3, 7, 4, having mesh of decreasing size according to the direction of filtering, of 2 mm and 0.6 mm, respectively, in the case of two sieves, and 2 mm, 1 mm and 0.6 mm, in the case of three sieves.

The filtering column 1 was agitated by means of a radial mixer 5 (Vortex Mixer) capable of guaranteeing a movement that is only oscillating and not sussultory and hence of allowing the adipose material treated to permeate the mesh of the sieves 3, 4 and 7 in an atraumatic manner. This mixer 5 guarantees almost total absence of traction and compression stresses and the total absence of centrifugal forces, which would cause stress of the cell material of the adipose tissue on the walls of the single sieves 3, 4 and 7 of the filtering column 1.

Moreover, in order to make accumulation of the valuable fraction T2a of the adipose tissue T1 atraumatic, after passing through the sieves (two or three) of the filtering column 1 a cylindrical shaped collection chamber 6 equipped with a flat bottom (or with a slight slope) was provided, such as to prevent a mechanical action of the upper layers of the cell material of the valuable fraction T2a of the adipose tissue T1, in relation to the lower layers, which would take place if it had a truncated-cone shaped section, which would cause a condition of stress thereto.

The vacuum dehydration-filter apparatus 2 was configured with a paper (or steel) filter 8, having a porosity between 20 and 25 micron, with a suction pump 9 and with an atmospheric air A inlet 11, associated with filtering valve means 23 for the air.

Regardless of the configuration of the filtering column 1, the oversize SP of the first sieve 3 (having mesh size of 2 mm) was sent to a treatment for disposal of the fibrous and/or oleic and/or haematic residues R1 deriving from the adipose tissue T1.

In the configuration of the filtering column 1 with two sieves, the undersize ST of the second sieve 4 (having a mesh size of 0.6 mm), accumulated in the collection chamber 6, was sent to the dehydration-filter apparatus 2 so as to obtain, finally, an adipose tissue T2a of Nanofat particle size. Moreover, the oversize SP of said second sieve 4 was collected, so as to obtain an adipose tissue T2b of Microfat particle size.

In the configuration of the filtering column 1 with 3 sieves, the oversize SP of the third sieve 7 (having a mesh size of 1 mm) was also collected, so as to obtain an adipose tissue T2c of Millifat particle size.

The vacuum dehydration-filter apparatus 2 determined separation of the aqueous residues R2 from the adipose tissue T2a by means of a vacuum pressure caused by a flow of filtered atmospheric air A, forced to move by the pump 9, from the upper section 10 toward the lower section 12 of the same apparatus. This vacuum pressure allowed the aqueous residues R2 of the adipose tissue T2a to pass through a filter 8 interposed between the upper section 10 and the lower section 12 of said apparatus and consequently to deposit in a collection tank 13.

The adipose tissue T2a, of Nanofat particle size, without fibrous and/or oleic and/or haematic residues R1 and aqueous residues R2, containing mesenchymal stem cells with a high survivability as they were subjected only to limited stress by the sieves 3, 4, 7 of the filtering column 1, was then collected from the filter 8 of the vacuum dehydration-filter apparatus 2 for possible reimplantation in the same donor patient, with methods and instruments of known type.

Likewise, the adipose tissue T2b, T2c, of Microfat and Millifat particle size, respectively, without fibrous and/or oleic and/or haematic residues R1, was collected from the sieves 4, 7 of the filtering column 1 for possible reimplantation in the donor patient, with methods and instruments of known type.

Said adipose tissue T2b, T2c can be collected directly from the sieves 4, 7 of the column 1, bypassing the vacuum dehydration-filter treatment, as:
it is naturally with a low water content, which tends to collect mainly in the undersize ST of the second sieve 4 of the filtering column 1;
having a particle size larger than Nanofat, the vacuum dehydration-filter treatment can cause increases in its density such as to make its reimplantation in the donor patient, by means of particularly thin cannulas, difficult.

### Example 2

Verifications of the method and of the device of the invention were carried out with the following methods of execution:
collection of a sample of adipose tissue T1 from a donor patient, by means of a cannula with a diameter of 2.2 mm, with lumen of 2 mm and 360° multiple holes of 1 mm, such as to reduce collection stress;
tumescent suction of the adipose tissue T1 subjected to collection, by means of 20 cc syringe;
regardless of the configuration of the filtering column 1, insertion of the adipose tissue T1 collected into the first sieve 3;
activation of the stirrer 5 for a stirring time of 1 minute with a rotation speed of 2000 rpm, these values being purely indicative and above all non-limiting for the purposes of implementation of the method or of use of the device of the invention;
after filtering, sending the fraction T2a of adipose tissue T1, accumulated in the collection chamber 6 to the vacuum dehydration-filter apparatus 2;
aspiration of the oversize of the filter 8 of the dehydration-filter apparatus 2 and sending it to a test laboratory to perform tests on the suitability for reimplantation of said fraction T2a of said adipose tissue T1.

Naturally, in real conditions of use, sending the fraction T2a of the adipose tissue T1 to the laboratory will be substituted by its reimplantation in the same donor patient, with known techniques and instruments.

### Results Obtained

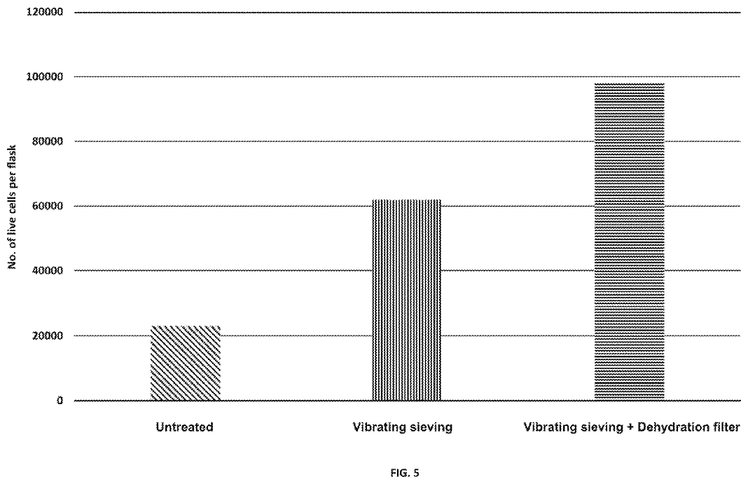

Below is a brief comment on the results obtained, indicated in the graphs of FIGS. 5-6-7 and in Tables T1 and T2, which describe the markers of the samples and the points in which it was sampled.

TABLE 2
Table T1

| Sample name | Explanation |
|---|---|
| Untreated | Sample collected from suction cannula Ø = 2 mm |
| Out (2set) | Sample collected after treatment with column |
| NO HA | configuration with 2 sieves (2 mm + 0.6 mm) |
| Out (3set) | Sample collected after treatment with column |
| NO HA | configuration with 3 sieves (2 mm + 1 mm + 0.6 mm) |
| Out (3set) | Sample collected after treatment with column |
| PRE HA | configuration with 3 sieves (2 mm + 1 mm + 0.6 mm) |
| | with the addition of HA upstream of the selection |
| | treatment. |
| Out (3set) | Sample collected after treatment with column |
| POST HA | configuration with 3 sieves (2 mm + 1 mm + 0.6 mm) |
| | with the addition of HA downstream of the |
| | selection treatment (before filtration). |

Simple observations in the syringe of an example of sample collected and subsequently processed give initial macroscopic evidence of homogeneity and elimination of haematic and fibrous residues from the adipose tissue. Examination under the optical microscope confirms this preliminary observation.

The graph in FIG. 5 shows how the method of the invention is capable not only of preserving the integrity and viability of the cells present in the adipose tissue collected but also of increasing their concentration by means both of cell selection and reduction of liquid residues. The first step of the method of the invention (filtering with vibrating sieving) has shown to be capable of suitably minimizing the cell stress in the steps aimed at restrictive collection of the final product. The triplicated number of viable cells at the end of this step is successfully obtained through the combination of debridement and of refining of the sample collected. The second step of the method of the invention (dehydration-filter) is an integral and fundamental part, as reduction of the excess liquid not only does not impair the performance of the first step but, on the contrary, it optimizes it, emphasizing the final data.

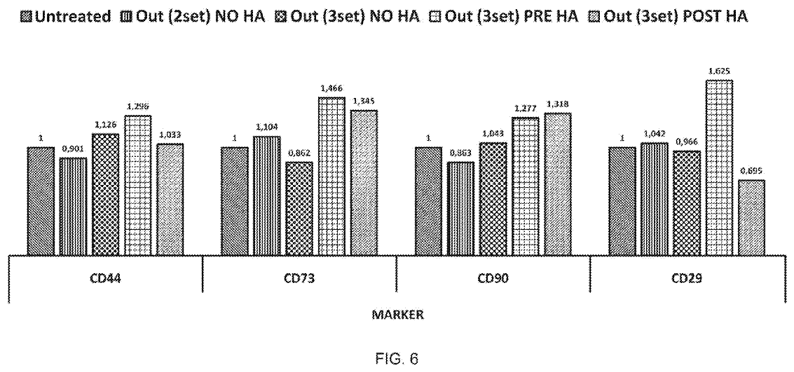

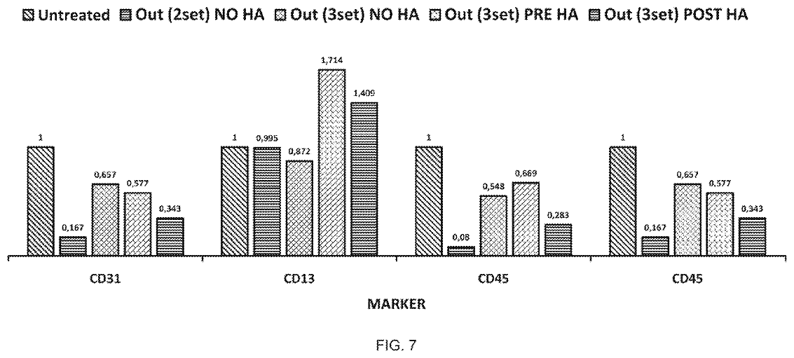

The graphs in FIGS. 6-7 shown how in all the samples examined all the cells express the markers sought and illustrate the synergy obtained with hyaluronic acid. The datum, evident and always reproducible, is represented by the main mesenchymal cells identified by the markers CD73, CD90, CD29.

The absence of haematopoietic/macrophage stem cells bears witness to the effective elimination of haematic residues. The adipocyte markers are poorly expressed as selection of the mesenchymal cells has been optimized while maintaining an adipose support for the stromal vascular niches. The quality of the population is given by the almost non-existent response of CD45, which indicates optimized elimination of mature cells.

The population must thus be considered highly homogeneous for mesenchymal (fibroblastoid) stem cells.

1. Method for treating an adipose tissue comprising the step of:
collecting an adipose tissue (T1) from a donor patient, characterized in that comprises the steps of:
providing a plurality of sieves with progressively decreasing mesh sizes;
providing radial stirrer means of oscillating type;
providing vacuum dehydration-filter means;
subjecting said adipose tissue (T1) to a first physical-mechanical treatment step by means of said plurality of sieves with progressively decreasing mesh size and by means of said radial stirrer means of oscillating type, separating at least an oversize fraction containing fibrous fractions of different particle sizes and a remaining wet and homogenized undersize fraction, of Nanofat type, rich in stem cells;
subjecting at least said remaining wet and homogenized undersize fraction to a second physical treatment step, by means of a vacuum dehydration-filter treatment, to eliminate the water, in order to concentrate the same fraction and obtain an adipose tissue (T2a) of Nanofat particle size, without fibrous and/or oleic and/or haematic residues (R1) and aqueous residues (R2), containing viable mesenchymal stem cells in a high concentration, where said adipose tissue (T2a) is adapted to be destined for reimplantation in the same donor patient.

2. Method according to claim 1, characterized in that said physical-mechanical treatment step comprises the steps of:
providing a filtering column (1), provided with at least a first and a second sieve (3, 4) placed in sequence, with mesh of decreasing size according to the direction of the filtering flow;
operating said filtering column (1) by means of said radial stirrer means (5) of oscillating type;
carrying out an impurity separation and homogenization treatment on the adipose tissue (T1) by means of the first sieve (3), obtaining an oversize (SP) and an undersize (ST);
disposing of the fibrous and/or oleic and/or haematic residues (R1) contained in the oversize (SP) of the first sieve 3;
carrying out an impurity separation and homogenization treatment, by means of the second sieve (4), on the undersize (ST) of the sieve immediately prior to it to obtain an oversize fraction (T2b) and a wet undersize fraction (ST) comprising Nanofat adipose tissue (T2a) and aqueous residue (R2).

3. Method according to claim 1, characterized in that said physical treatment step comprises the steps of:
providing a vacuum dehydration-filter apparatus (2), provided with a filter (8);
providing a suction pump (9) adapted to operate said vacuum dehydration-filter apparatus (2);
carrying out a separation treatment of aqueous residues (R2) from said remaining wet and homogenized undersize fraction (ST) by means of the filter (8) and the suction pump (9) of the vacuum dehydration-filter apparatus (2), so as to obtain an adipose tissue (T2a) of Nanofat particle size, also without aqueous residues (R2).

4. Method according to claim 2, characterized in that comprises the further step of:
collecting an oversize (SP) of the second sieve (4), comprising an adipose tissue (T2b) of Microfat particle size, naturally with a low water content and without fibrous and/or oleic and/or haematic residues (R1), containing viable mesenchymal stem cells in a high concentration, where said adipose tissue (T2b) of Microfat particle size is adapted to be destined for reimplantation in the same donor patient.

5. Method according to claim 2, characterized in that comprises the further steps of:
providing a third sieve (7), interposed between the first and the second sieve (3, 4) obtaining a further oversize (SP) and a further undersize (ST);
by means of the third sieve (7), carrying out an impurity separation and a subsequent homogenization treatment on the undersize (ST) of the sieve immediately prior to it;
collecting the oversize (SP) of the third sieve (7), so as to obtain an adipose tissue (T2c) with Millifat particle size, naturally with a low water content and without fibrous and/or oleic and/or haematic residues (R1), containing viable mesenchymal stem cells in a high concentration, destined for reimplantation in the same donor patient.

6. Device for treating an adipose tissue (T1), collected from a donor patient, characterized in that it comprises:
a filtering column (1), provided with at least a first and a second sieve (3, 4), with mesh of decreasing size according to the direction of the filtering flow, adapted to be operated by radial stirrer means (5) of oscillating type;
a vacuum dehydration-filter apparatus (2), provided with a filter (8), adapted to be operated by a suction pump (9),
where said filtering column (1), by means of said second sieve (4), allows an undersize (T2a) and aqueous residue (R2) and comprising a wet adipose tissue of Nanofat particle size and an oversize comprising an adipose tissue (T2b) of Microfat particle size, naturally with a low water content, to be obtained and where said vacuum dehydration-filter apparatus (2) allows an adipose tissue (T2a) without aqueous residues (R2) to be obtained, where each adipose tissue (T2a), (T2b) is without fibrous and/or oleic and/or haematic residues (R1), contains viable mesenchymal stem cells in a high concentration, and at least one of these is destined for reimplantation in the same donor patient.

7. Device according to claim 6, characterized in that the first and the second sieve (3, 4) comprise filtering mesh of 2 mm and of 0.6 mm in size, respectively.

8. Device according to claim 6, characterized in that the filtering column (1) comprises:
a reversible closing cover (20) of the first sieve (3), provided with sealed points (21) for insertion of the adipose tissue (T1) optionally in combination and/or dosage with substances having therapeutic action;
a collection chamber (6) of the undersize (ST) of the second sieve (4), equipped with a flat bottom or with a weak slope.

9. Device according to claim 6, characterized in that the filtering column (1) further comprises:
a third sieve (7), arranged in intermediate position between the first and the second sieve (3, 4),
where said third sieve (7) allows a further oversize to be obtained comprising an adipose tissue (T2c) of Millifat particle size, naturally with a low water content and without fibrous and/or oleic and/or haematic residues (R1), containing viable mesenchymal stem cells in a high concentration, destined for reimplantation in the same donor patient.

10. Device according to claim 9, characterized in that the third sieve (7) defines the central portion of the filtering column (1).

11. Device according to claim 9, characterized in that the third sieve (7) has a mesh size of 1 mm.

12. Device according to claim 6, characterized in that the vacuum dehydration-filter apparatus (2) comprises:
an upper section (10), provided with a reversible closing cover (22);
an atmospheric air (A) inlet (11), positioned on the closing cover (22);
one-way filtering valve means (23) associated with the inlet (11), adapted to filter atmospheric air (A);
a lower section (12), provided with a tank (13) for collecting aqueous residues (R2), separated from the undersize (ST) of the second sieve (4) of the filtering column (1).

13. Device according to claim 6, characterized in that the filter (8) of the vacuum dehydration-filter apparatus (2) is made of paper or of steel, with porosity between 20 and 25 micron.
